(19) United States
(12) Patent Application Publication
(10) Pub. No.: US 2026/0218102 A1
(43) Pub. Date: Jul. 30, 2026

(54) BIOREACTOR BAG AND SYSTEMS AND METHODS OF USING SAME

(71) Applicant: METABOGAL SCIENTIFIC CONSULTING LTD., Timrat (IL)

(72) Inventors: Yoseph SHAALTIEL, Timrat (IL); Yacob GILAN, Binyamina (IL)

(21) Appl. No.: 19/146,199

(22) PCT Filed: Jan. 23, 2024
(86) PCT No.: PCT/IL2024/050091

§ 371 Date: Jul. 7, 2025

Related U.S. Application Data
(60) Provisional application No. 63/441,237, filed on Jan. 26, 2023.

(51) Int. Cl. C12M 1/00; C12M 1/02

(52) U.S. Cl. CPC C12M 23/14; C12M 23/50; C12M 27/00; C12M 29/00

(57) ABSTRACT

A bioreactor system including a pliable bag for containing a liquid, an air inlet port positioned at a top portion of said pliable bag and one or more tubes connected to the inlet port and running along a height and/or width of the bag. The tube(s) has a closed distal end and a plurality of openings running a length thereof with a configuration and/or diameter of each of the openings selected such that flow of air through the openings is substantially equal along the length of the tube.

### BACKGROUND

The present invention relates to bag-type bioreactor and to systems and methods of using same for growing plant cells, algae, fungi filaments and bacteria. Embodiments of the present invention relate to a bioreactor that is configured for enhanced aeration and a bioreactor system configured for enhanced harvesting and filling.

The demand for biologics has increased in recent years in both the biopharmaceutical and enzymatic manufacturing industries as well as the food industry. Such demand has driven the need to process higher volumes and as such, an increasing number of manufacturers are turning to bag-type bioreactors that are typically configured as a single use solution.

Instead of a culture vessel made from stainless steel or glass, a bag-type bioreactor is usually made from a multi-layer polymer sheet that is welded or glued to form a cylindrical bag having a volume of several hundred liters. The bag is fitted with air inlet, exhaust and harvesting/filling ports and with internal stirrers or external agitators.

Compared with conventional bioreactor systems, bag-type bioreactors eliminate cleaning and reduce sterilization demands and the risk of cross contamination while enhancing biological and operational safety. In addition, bag-type bioreactors are cheaper to produce, contain fewer parts than conventional bioreactors and as a result reduce initial as well as ongoing operating costs.

While bag-type bioreactors are well known in the art and have been used in culturing of mammalian as well as plant cells and algae for decades, presently used configurations typically bubble oxygen from the bottom and as such, the top part of the bioreactor culture media is typically less saturated with oxygen. This poses a limit on the growth and productivity of the cultured organism and the concentration of proteins or secondary metabolites produced thereby.

Another limiting feature of presently used bag-type bioreactors are the filling and harvesting ports. Due to their configuration, opening and closing such ports is typically a time consuming, labor-intensive process that is sensitive to contamination. There is thus a need for, and it would be highly advantageous to have, a bag-type bioreactor system devoid of the above limitations.

### SUMMARY

According to one aspect of the present invention there is provided a bioreactor system comprising a pliable bag for containing a liquid, an air inlet port positioned at a top portion of the pliable bag and at least one tube connected to the inlet port and having a closed distal end positioned at a bottom portion of the pliable bag, the tube having a plurality of openings running a length thereof with a configuration and/or diameter of each of the openings being selected such that flow of air through the openings is substantially equal along the length of the tube when the tube is positioned within a liquid contained within the pliable bag.

According to embodiments of the present invention a diameter of the openings increases along the length from the top portion to the bottom portion of the pliable bag.

According to embodiments of the present invention a diameter of the openings increases along the length according to:

$$Q = \frac{1}{2} π {r}^{4} \frac{Δ p}{8 η L} ,$$

wherein Q=volume of flow per unit time, r=radius of the tube, ΔP=pressure drop across the tube, L=length of the tube, n=viscosity (Pas).

According to embodiments of the present invention the pliable bag is sized for containing 50-2000 Liters of the fluid.

According to embodiments of the present invention the pliable bag is fabricated from polyethylene, polycarbonate or PVC.

According to embodiments of the present invention the air inlet tube is 50-250 cm in length and 2-20 mm in internal diameter.

According to embodiments of the present invention the bioreactor further comprises a filling and harvesting port positioned at a bottom end of the pliable bag.

According to embodiments of the present invention a distal end of the tube is maintained in a position within the pliable bag via magnets.

According to embodiments of the present invention the filling and harvesting port includes a first valve assembly being operable by a second valve assembly of an external harvesting and filling unit, such that both the first valve assembly and the second valve assembly open upon engagement to enable harvesting or filling of the pliable bag.

According to another aspect of the present invention there is provided bioreactor system comprising: a pliable bag for containing a liquid, an air inlet port positioned at a top portion of the pliable bag and at least single tube connected to the inlet port and having a closed distal end positioned at a bottom portion of the pliable bag, the tube having a plurality of air openings running a length thereof wherein a distal end of the tube is maintained in a position within the pliable bag via magnets.

According to another aspect of the present invention there is provided a bioreactor system comprising: a pliable bag for containing a liquid and a first valve assembly positioned at a bottom of the pliable bag, the first valve assembly being operable by a second valve assembly of an external harvesting and filling unit, such that both the first valve assembly and the second valve assembly open upon engagement to enable harvesting or filling of the pliable bag.

According to one aspect of the present invention there is provided bioreactor system comprising: a pliable bag for containing a liquid, the pliable bag having a valve assembly positioned at a bottom thereof, a support structure for suspending the pliable bag from an anchor point and a mobile harvesting and filling unit for engaging the valve assembly to enable harvesting and filling the pliable bag.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-B illustrate front (FIG. 1A) and isometric (FIG. 1B) views of one embodiment of the present bioreactor.

FIGS. 2A-F illustrate front (FIG. 2A, C, E) and side (FIG. 2B, D, F) views of embodiments of the present bioreactor having different aeration tube configurations.

FIG. 3 illustrates the harvesting/filling valve assembly in a closed position with the bioreactor portion on top and the harvesting/filling unit portion on the bottom.

FIG. 4 illustrates the bioreactor (bag) portion of the harvesting/filling valve in an open position.

FIG. 5 illustrates the harvesting/filling unit portion of the harvesting/filling valve in an open position.

### DETAILED DESCRIPTION

The present invention is of a bag-type bioreactor which can be used for cell culturing. Specifically, the present invention can be used to culture native or modified cells for the purpose of producing biomolecules.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Bag-type (pliable container) bioreactors are well known in the art and find increasing use in large scale culturing of cells. While such bioreactors have several advantages over standard (rigid container) bioreactors, there are still some drawbacks that prevent widespread use of such culturing systems.

While reducing the present invention to practice, the present inventor set out to improve bag-type bioreactors by enhancing bag aeration and harvesting/filling thereby potentially increasing output and decreasing operating turnaround time.

Thus, according to one aspect of the present invention there is provided a bioreactor system that can be used to culture cells such as, plant cells, bacteria, fungi filaments, mammalian cells and the like for the purpose of harvesting the cells or a product thereof. The cells can be native cells that can be harvested for cell components such as secondary metabolites, fatty acids and/or polysaccharides or they can be genetically-modified cells that produce biologics that can be used in the pharmaceutical, enzyme-manufacturing industries and food industry. Examples of biologics include polymers, proteins, polynucleotides, polysaccharides, and the like.

The bioreactor system includes a pliable bag for containing a liquid such as a culture media. The bag can be configured as a cylinder when filled with a volume of 10-2000 liters. Typical dimensions of the bag can be 50-250 cm height, 10-100 cm width, 0.2-0.4 cm thickness (front to back) when empty and 6.36-63.6 cm thickness when filled. The bag can be fabricated from single or multi-layer sheets of a polymer or polymers using welding or bonding technologies.

The reactor can be made from two polymer sheets welded at the perimeter and the bottom to form a cone. The bottom end of the cone can include a welded flange for fitting the harvesting and filling valve. A second flange can be welded at the top for the aeration port.

The bag can include mounts (e.g., reinforced eyelets) at the top for hanging the bag from a ceiling fixture. The bioreactor system can include a hammock or sling for supporting the bag or alternatively, the bag can include an integrated support structure that can be fabricated from metal wires or polymeric straps. Such wires or straps can form a mesh or a sling that can be positioned (optionally bonded) between the layers of the bag or bonded to an external surface thereof.

The bag can include an air inlet port positioned at a top with a tube or several tubes connected to the inlet port and running along the side of the bag (internally) from top to bottom. The bottom end of the tube(s) can be maintained in position via a pair of magnets, one fitted to the tube (e.g., as a plug to the bottom opening of the tube) while the other fixed outside the bag. The magnets can be neodymium magnets grade N30-N52.

The tube can be 30-300 cm in length, 2-20 mm in external diameter and 1-18 mm in internal diameter. The distal end of the tube positioned at a bottom of the bag is closed and a plurality of air openings run the length thereof with a configuration and/or diameter of each of the openings being selected such that flow of air through the openings is substantially equal along the length of the tube.

For example, the diameter of the openings can increase along the length of the tube from top to bottom according to the following formula:

$$Q = \frac{1}{2} π {r}^{4} \frac{Δ p}{8 η L} ,$$

wherein Q=volume of flow per unit time, r=radius of the tube, ΔP=pressure drop across the tube, L=length of the tube, n=viscosity (Pas).

Alternatively, maintaining equal aeration along the length of the tube can be achieved by varying the number of openings covering each centimeter of length.

Regardless of approach, maintaining equal air flow along the length of the tube is important in order to aerate the reactor evenly. Lack of well distributed oxygen concentration within the reactor is the cause for cell stress, low growth and low productivity.

The bag can be fitted with an exhaust port at the top for releasing air pressure from the bag and a harvesting/filling port at a bottom end of the bag for filling the bag with culture media and cells and for collecting cells and/or culture media.

The harvesting/filling port can include a first valve assembly that is operable by a second valve assembly of an external harvesting and filling unit. When the first valve assembly and the second valve engage, both open to enable harvesting or filling of the pliable bag from the external harvesting and filling unit. Such a dual-opening configuration is advantageous in that it enables both harvesting and filling from a single port that is easily sterilizable. A dual-function port allows simple automation of the system enabling harvesting and filling of large number of reactors quickly and easily.

Both the first and second valve assemblies can each include a spring-loaded piston attached to a cap sealing a cylinder within which the piston resides. Upon engagement, the pistons of the two valve assemblies translate within their respective cylinders in opposite directions thereby uncovering the openings of the two connected cylinders and opening the harvesting/filling port.

The first and second valve assemblies can also include a hole in a sidewall thereof to enable access to the internal conduit of the valves and thereby enable sterilization thereof.

The bag can alternatively include other harvesting/filling port configurations (e.g., top mounted filling port and a bottom mounted harvesting port) that can be accessed via an external unit or manually and a top or bottom inlet for aeration (via bubbling).

Alternatively, the dual-opening configuration described above can be used with other aeration configurations (e.g., several aeration ports at different depths of the bag).

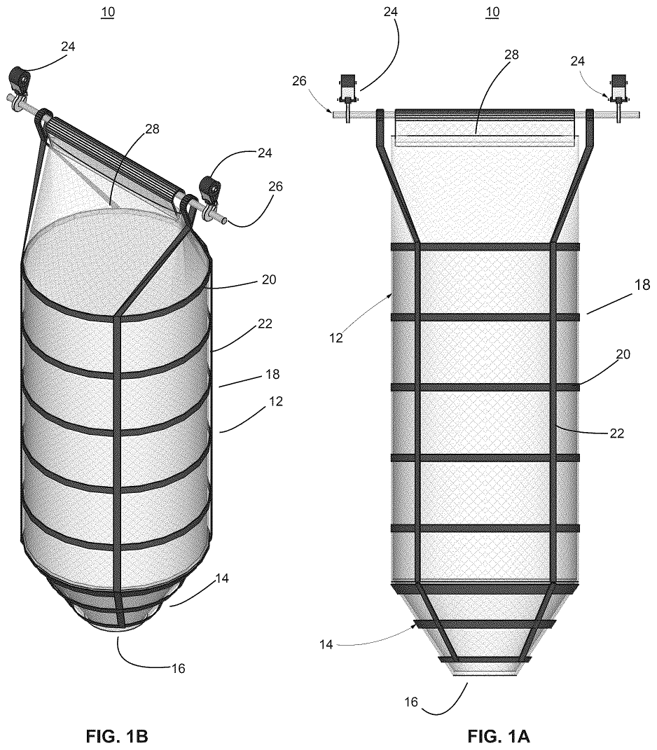

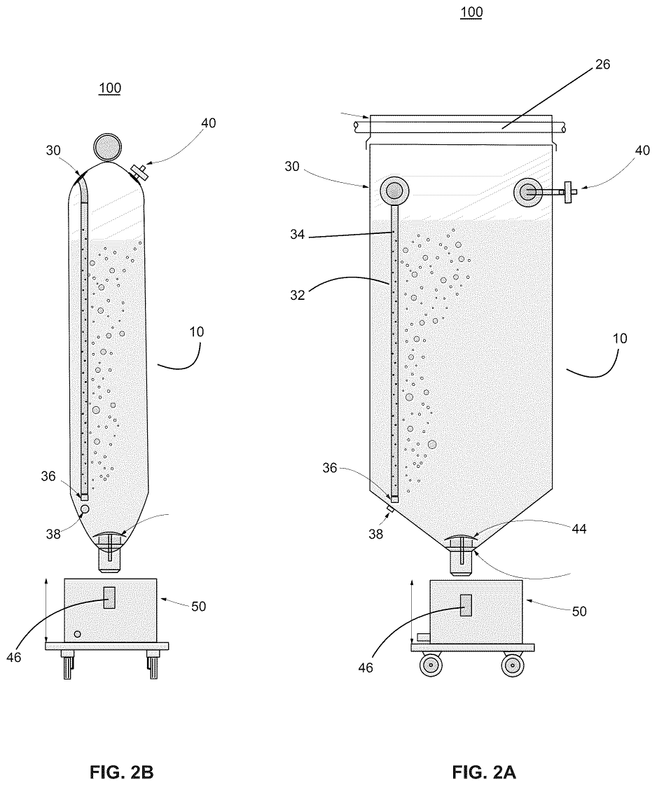

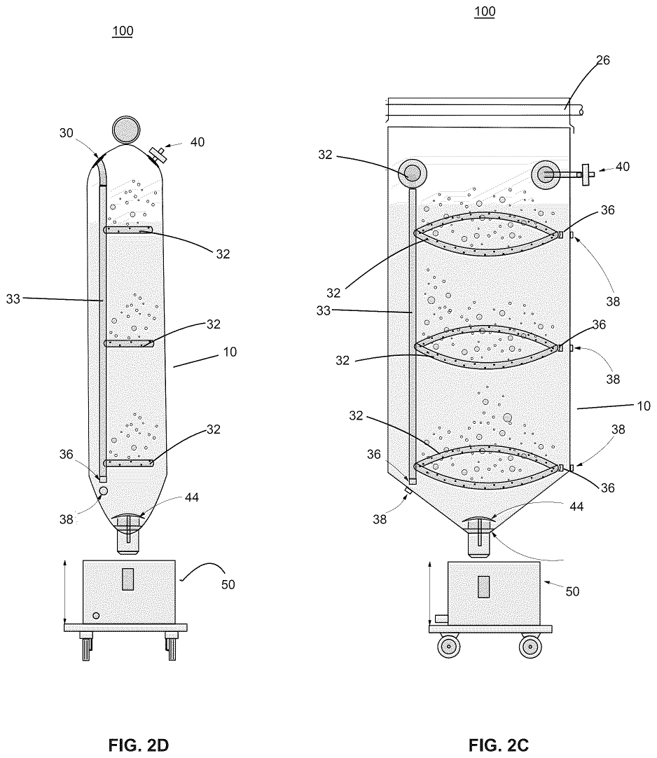

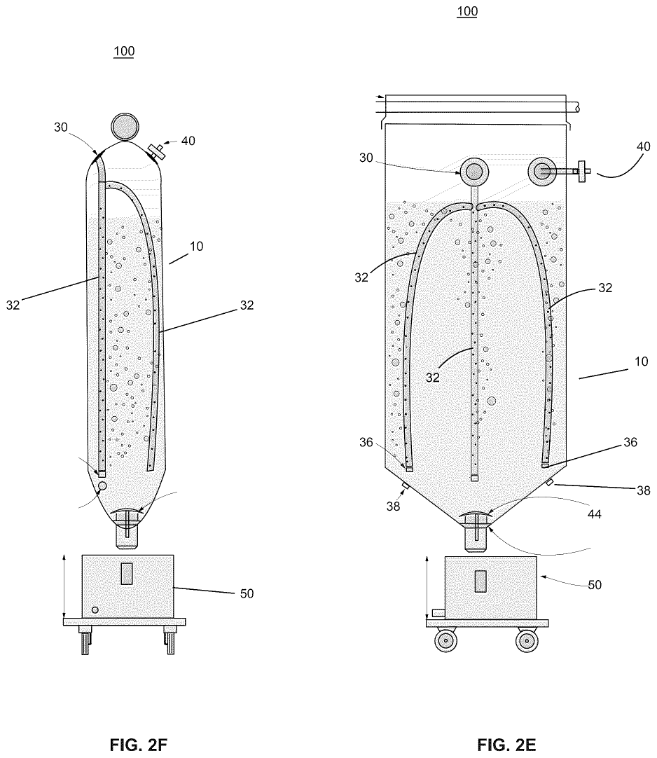

Referring now to the drawings, FIGS. 1A-B illustrate the bag of the present bioreactor system which is referred to herein as bag 10. FIGS. 2A-B illustrate a system that includes bag 10 (referred to herein as system 100) and an external harvesting/filling unit (referred to herein as unit 50). The configuration of system 100 described hereinunder is suitable for culturing native or genetically modified plant cells and harvesting a biomolecule (e.g., protein product) from the cells and/or culture medium. It will be understood however, that one of ordinary skill in the art would be capable of applying system 100 with modifications to culturing of any cell line for any purpose.

As is shown in FIGS. 1A-B, when filled with media bag 10 forms a cylindrical body 12 that conically tapers through a bottom portion 14. The height of bag 10 can be 50-200 cm, the diameter of body 12 can be 10-100 cm and the diameter at a bottom end 16 can be 5 cm.

Bag 10 can be fabricated from polyethylene polymer layers either sheets that can be welded or polyethylene cylinder that can be welded to a conical shape at the bottom.

Bag 10 can include a support structure 18 that is removably attached and can be reused or optionally bonded to the outer surface of bag 10. Support structure 18 can include, for example, 8 circumferential straps 20 attached to 4 longitudinal straps 22. Straps 20 and 22 can be fabricated from woven nylon or similar polymers, in case it is bonded to the surface of bag 10 it could be welded during bag manufacturing.

Bag 10 and support structure 18 can be hung from ceiling fixtures 24 (e.g., hooks) through a rod 26 by looping longitudinal straps 22 and a top flap 28 of bag 10 over rod 26. While bag 10 can be hung on rod 26 without support structure 18, the latter provides extra support and ensures that bag 10 does not rupture during culturing or transport.

As is shown in FIGS. 2A-F, bag 10 includes ports for aeration, exhaust, and harvesting/filling.

Port 30 is configured for aerating the contents of bag 10. Port 30 is formed by welding a connection through a circular opening formed in the wall of bag 10.

Port 30 is connected to at least one tube 32 (one shown in FIGS. 2A-B and four in FIGS. 2C-F) that runs vertically from port 30 to a bottom region in bag 10 (FIGS. 2A-B), forms four horizontal crescents each connected to a feed tube 33 (FIGS. 2C-D) or three vertical tubes 32 that form a claw-like configuration (FIGS. 2E-F). Tube 32 can be any length with an internal diameter of 1-10 mm. Tube 32 includes a plurality of openings 34 that vary in diameter from top to bottom (in the vertical configurations). For example, tube 32 can include 20 circular openings that gradually vary from 0.1 mm in diameter at a top to 1 mm in diameter at a bottom. Openings 34 can be arranged circumferentially around tube 32 or on a single face thereof (pointing away from a wall of bag 10).

FIGS. 2A-B, 2C-D and 2E-F illustrate three different aeration configurations of system 10. FIGS. 2A-B illustrate a single tube 32 aeration configuration that runs from top to bottom of bag 10. FIGS. 2C-D illustrate a vertical tube 33 (that may or may not include aeration holes) connected to three horizontal tubes loops that have aeration holes. FIGS. 2E-F illustrate a configuration having four interconnected vertical tubes 32. The embodiments shown in FIGS. 2C-F can enable a more robust and rapid aeration when growing high demanding organisms like bacteria or organisms that can tolerate high shear forces.

Tube(s) 32 can be maintained in position via magnets. A first magnet 36 can be positioned at, for example, a bottom opening of tube 30 (effectively plugging tube 30) while a second magnet 38 can be positioned on an outer surface of bag 10.

Port 30 is connectable to a source of air (e.g., pump or air condenser with 0.2μ filter) and via tube 30 can provide an air flow of 10-200 ml/min.

Bag 10 is also fitted with an exhaust port 40 for removing excess air from bag 10. Exhaust port can be made of a silicon tube either open or fitted with 0.2μ air filter.

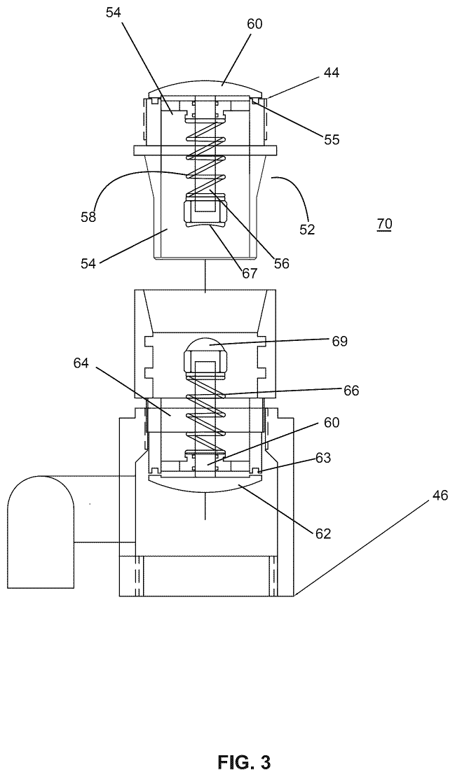

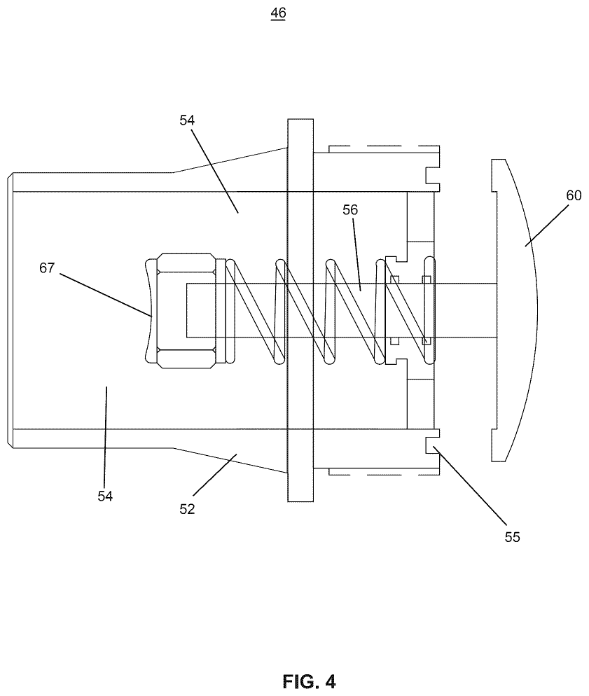

Bag 10 is also fitted with a harvesting/filling port 42 that includes a first valve assembly 44 configured for engaging a second valve assembly 46 (FIGS. 3 and 4B) positioned within unit 50. First valve assembly 44 can include a flange that is bonded or welded to a circular opening at the reactor bottom.

Unit 50 can be an autonomous or remotely controlled cart that can navigate underneath bag 10 to engage port 42 for harvesting of filling of bag 10. Unit 50 can be a cart or trolly having a top fitted with a harvesting port. The trolly can be equipped with a video navigation system to follow a marked path to and from bag 10 and sensors (e.g., image) for identifying first valve assembly 44 of bag 10 in order to harvest and fill bag 10.

Unit 50 can also be a stationary unit in which case bag 10 can be maneuvered over unit 50 using a motorized ceiling conveyer system. In such a configuration the conveyer system identifies unit 50 and positions port 42 thereover. The conveyer system can either lower bag 10 to engage first valve assembly 44 with second valve assembly 46 or unit 50 can rise to enable such engagement.

Unit 50 can also include a port for connecting to a source of a sterilizing agent (e.g., steam or disinfecting agent) and optionally an additional port for connecting to an external tank via a flexible hose. The external tank can include culture media or it can be used to store the harvested media and/or cells. The external tank can include a pump for harvesting or filling of bag 10 through unit 50.

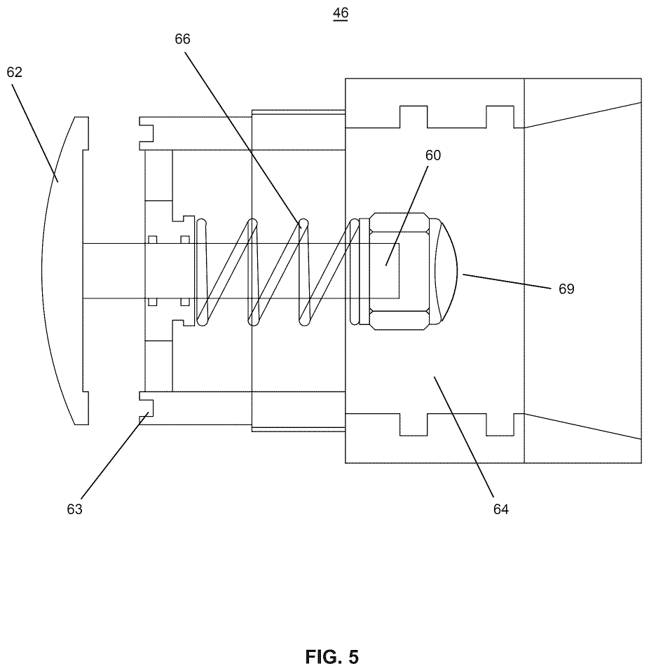

FIGS. 3 and 4-5 illustrate first valve assembly 44 (port 42) and second valve assembly 46 (respectively) in greater detail.

First valve assembly 44 includes a valve body 52 that includes a cylindrical bore 54 having a piston 56 disposed therein and on top of spring 58. Piston 56 is attached to a cap 60 that covers and seals (via O-ring 55) an opening of cylindrical bore 54.

First valve assembly 44 is shown in a closed position in FIG. 3 with spring 58 uncompressed. When engaged with second valve assembly 46 (at 67 and 69, FIG. 3), piston 56 is pushed up to disengage cap 60 from the opening of cylindrical bore 54 thereby opening first valve assembly 44. Spring 58 is compressed (loaded) such that when first valve assembly 44 disengages from second valve assembly 46, piston 56 moves back down to seal cylindrical bore 54 and close first valve assembly 44.

Second valve assembly 46 has a similar configuration with piston 60 (fitted with cap 62 over O-ring seal 63) positioned within cylindrical bore 64 and over spring 66. Engagement between first valve assembly 44 and second valve assembly 46 (at 67 and 69, FIG. 3) pushes piston 60 down and compresses spring 66 to open second valve assembly 46. Disengagement releases spring to close second valve assembly 46. First valve assembly 44 and second valve assembly 46 can each include an opening 70 into cylindrical bores 54 and 64 (respectively) for allowing sterilization thereof.

A culture media is prepared according to the grown organism in an external vessel (bag or stainless steel vat). The culture media and cells are then pumped into bag 10 through unit 50 via the harvesting filling port. Once bag 10 is full, unit 50 is detached to close off valves 44 and 46.

As used herein the term “about” refers to +10%.

Reference is now made to the following examples, which together with the above descriptions, illustrate the invention in a non limiting fashion.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1.-13. (canceled)

14. A bioreactor system comprising:
(a) a pliable bag for containing a liquid;
(b) an air inlet port positioned at a top portion of said pliable bag; and
(c) at least one tube connected to said inlet port and having a closed distal end positioned at a bottom portion of said pliable bag, said tube having a plurality of openings running a length thereof with a configuration and/or diameter of each of said openings being selected such that flow of air through said openings is substantially equal along said length of said tube when said tube is positioned within a liquid contained within said pliable bag.

15. The bioreactor of claim 14, wherein a diameter of said openings increases along said length from said top portion to said bottom portion of said pliable bag.

16. The bioreactor of claim 14, wherein a diameter of said openings increases along said length according to:
$$Q = \frac{1}{2} π {r}^{4} \frac{Δ p}{8 η L} ,$$
wherein Q=volume of flow per unit time, r=radius of said tube, ΔP=pressure drop across said tube, L=length of said tube, η=viscosity (Pas).

17. The bioreactor of claim 14, wherein said pliable bag is sized for containing 50-1000 L of said fluid.

18. The bioreactor of claim 14, wherein said pliable bag is fabricated from polyethylene, polycarbonate or PVC.

19. The bioreactor of claim 14, wherein said tube is 50-250 cm in length and 2-20 mm in internal diameter.

20. The bioreactor of claim 14, further comprising a filling and harvesting port positioned at a bottom end of said pliable bag.

21. The bioreactor of claim 14, wherein a distal end of said tube is maintained in a position within said pliable bag via magnets.

22. The bioreactor of claim 20, wherein said filling and harvesting port includes a first valve assembly being operable by a second valve assembly of an external harvesting and filling unit, such that both said first valve assembly and said second valve assembly open upon engagement to enable harvesting or filling of said pliable bag.

23. A bioreactor system comprising:
(a) a pliable bag for containing a liquid;
(b) an air inlet port positioned at a top portion of said pliable bag; and
(c) at least single tube connected to said inlet port and having a closed distal end positioned at a bottom portion of said pliable bag, said tube having a plurality of air openings running a length thereof wherein a distal end of said tube is maintained in a position within said pliable bag via magnets.

24. A bioreactor system comprising:
(a) a pliable bag for containing a liquid; and
(b) a first valve assembly positioned at a bottom of said pliable bag, said first valve assembly being operable by a second valve assembly of an external harvesting and filling unit, such that both said first valve assembly and said second valve assembly open upon engagement to enable harvesting or filling of said pliable bag.
